(19) United States
(12) Patent Application Publication
(10) Pub. No.: US 2022/0154126 A1
(43) Pub. Date: May 19, 2022

(54) SET OF ADHESIVE MAT AND PRESSURE AID FOR USE FOR LABORATORY SHAKERS OR SHAKING INCUBATORS

(71) Applicant: Thermo Electron LED GmbH, Langenselbold (DE)

(72) Inventors: Johann Naumann, Osterode (DE); Norman Ballhause, Osterode (DE)

(21) Appl. No.: 17/475,569

(22) Filed: Sep. 15, 2021

(30) Foreign Application Priority Data
Nov. 17, 2020 (DE) 102020214452.4

(51) Int. Cl. C12M 3/06; C09J 127/18; C12M 1/00; B01F 11/00; C09J 143/04

(52) U.S. Cl. CPC C12M 27/16; C09J 127/18; C09J 143/04; B01F 11/0014; C12M 41/14

(57) ABSTRACT

The present invention relates to a set comprising an adhesive mat for the shaking platform of a laboratory shaker or shaking incubator, the adhesive mat having a platform side for application to the shaking platform and a support side opposite the platform side, both of which are designed to be adhesive, the set comprising a pressure aid which has a contact surface which is anti-adhesive with respect to the support side of the adhesive mat. The present invention also relates to a use of a pressure aid of this type for the attachment of an adhesive mat to a shaking platform of a laboratory shaker or shaking incubator and to a method for the attachment of an adhesive mat to a shaking platform of a laboratory shaker or shaking incubator.

### CROSS-REFERENCE TO RELATED APPLICATION

The present application claims priority under 35 U.S.C. § 119 of German Patent Application No. 10 2020 214 452.4, filed Nov. 17, 2020, the disclosure of which is hereby incorporated herein by reference in its entirety.

### FIELD OF THE INVENTION

The present invention relates to a set comprising an adhesive mat for a shaking platform of a laboratory shaker or shaking incubator, the adhesive mat having a platform side for application to the shaking platform and a support side opposite the platform side, both of which are designed to be adhesive, and a pressure aid. In addition, the present invention relates to the use of a pressure aid for the attachment of an adhesive mat to a shaking platform of a laboratory shaker or shaking incubator and to a related method.

### BACKGROUND OF THE INVENTION

Laboratory shakers and incubators of the generic type are used in a large number of laboratories, for example in the field of chemistry, pharmacy, biotechnology, microbiology, and many other fields. They are offered both as small table-top devices and as large, free-standing devices. Devices of this type may have one or more shaking platforms or trays on which a large number of different containers, for example bottles, Erlenmeyer flasks, microtiter plates, etc., can be stored. The shaking platform is moved at a typically adjustable speed, for example linearly back and forth, rotating in an orbital movement or in a three-dimensional tumbling movement. As a result, the contents of the containers are mixed and/or kept in movement on the shaking platform. In the case of laboratory shakers, the shaking platform is exposed in the laboratory atmosphere or is covered by a lid. Shaking incubators, on the other hand, have an interior space surrounded by a housing, in which mostly a plurality of separately or jointly moving shaking platforms is arranged and in which controlled conditions such as a specific temperature and/or humidity and possibly a specific gas composition prevail. Laboratory shakers and shaking incubators of the generic type are described, for example, in EP 1201297 A1 , WO 2005/107931 A1, EP 1445307 A1, and EP 1949955 A1 and are sold by Thermo Fisher Scientific, Inc. under the names “Fisherbrand™ Schüttler,” “Thermo Scientific™ Solaris™,” and “Thermo Scientific™ MaxQ™.”

It is known to provide the shaking platforms of generic devices with different attachments in order to be able to advantageously attach different containers to the shaking platforms, for example by means of clamps, holders, and webs. In addition, so-called adhesive mats are known which are applied to the shaking platforms and serve as a non-slip base for containers and other equipment placed on the shaking platform. Generic adhesive mats are typically resilient, flat bases which are a few millimeters thick, which are usually made of a plastics material. The two surfaces are usually permanently adhesive. For example, the surfaces can each have a layer of permanently adhesive pressure-sensitive adhesive, in particular based on a polyacrylate dispersion. Adhesive mats of this type are available, for example, from Thermo Fisher Scientific Inc. as “Adhesive Flask Mat” or under the names “Sticky Stuff” from Infors AG and “IKA Stickmax” from IKA-Werke GmbH & Co. KG.

Due to the permanently adhesive properties, the adhesive mat adheres to the material of the shaking platform (mostly metal, in particular stainless steel, glass, or plastics material). Containers or the like placed on the support side (upper side) of the adhesive mat also adhere to the surface of the adhesive mat and are thus protected against slipping, even when the shaking platform is moving. Due to the strongly adhesive properties of the adhesive mat, however, there are some practical problems when applying the adhesive mat to the shaking platform. Due to the adhesive properties of the adhesive mat, it adheres or sticks not only to the shaking platform itself, but also, for example, to the fingers or gloves of the person who wants to attach the adhesive mat to the shaking platform. In order to attach the adhesive mat to the shaking platform in as flat and even a manner as possible, it must be pressed on the shaking platform without air inclusions and formation of wrinkles. However, this is difficult because of the strong adhesive force of the adhesive mat. Often, the adhesive mat is moistened before it is attached to the shaking platform, for example by immersion in water, in order to temporarily reduce the adhesiveness and to simplify the attachment of the adhesive mat to the shaking platform. However, this measure means that the adhesive mat first has to dry before the shaker or shaking incubator can be put into operation, since otherwise there is a risk that the adhesive mat will slip again or that the containers placed thereon will not stand securely. However, this wastes valuable time in which the devices cannot be used. Since the adhesive force of the adhesive mats decreases over time, for example because dust particles occupy the surface of the adhesive mats, it is recommended that they be removed regularly from the shaking platforms and washed with a surfactant solution to clean the surfaces. When reapplying the adhesive mat to the shaking platform, the adhesive mat then has to be dried again, which causes further loss of time. In addition, the adhesion of the adhesive mat to the shaking platform in the case of a wet-applied adhesive mat, despite thorough drying, is often less than the adhesion of the same adhesive mat if it was applied to the shaking platform without moistening.

It is therefore an aspect of the present invention to simplify the attachment of an adhesive mat to a shaking platform, in particular to reduce the time required for this, and to improve the adhesion thereof.

### SUMMARY OF THE INVENTION

Specifically, the solution is achieved in the case of a set described at the outset in that, in addition to an adhesive mat, it comprises a pressure aid which has a contact surface that is anti-adhesive with respect to the support side of the adhesive mat. The side of the adhesive mat that comes into contact with the shaking platform when it is placed on the platform, i.e., the underside from the operator's point of view, is referred to below as the platform side. The support side denotes the side opposite the platform side; from the operator's point of view, the upper side and that side of the adhesive mat on which containers or the like to be shaken during operation of the shaking platform are placed. The pressure aid is therefore a tool that can be used by an operator for the attachment of the adhesive mat to the shaking platform. For this purpose, the pressure aid has a contact surface which is designed to come into contact with the adhesive mat and, in particular, the support side thereof, so that an operator can exert a force on the adhesive mat using the pressure aid via its contact surface, the adhesive mat being pressed onto the shaking platform and being fixed in place. “Fixed” means that the adhesive mat is detachably bonded to the shaking platform. The adhesive mat can therefore be detached from the shaking platform again with the use of force, while the position of the adhesive mat does not change relative to the shaking platform during normal shaking operation. A permanent attachment of the adhesive mat to the shaking platform, whereby the adhesive mat can no longer be detached from the shaking platform in a non-destructive manner, is in principle also conceivable, but not preferred within the scope of the present invention. A set according to the present invention can of course also contain more than just one adhesive mat, whereby the adhesive mats can be the same or different. For example, the adhesive mats can differ in size.

In principle, any prior art adhesive mat that has two adhesive surfaces (platform side and support side) can be used as the adhesive mat of the set according to the present invention. As described at the outset, these sides are expediently permanently adhesive, and the adhesive force on the two sides can be the same or different. In one variant, the adhesive mat consists of a uniform material. In a further variant, the adhesive mat has a resilient core film, which expediently consists of a resilient plastics material which is as chemically inert and thermally stable as possible, the core film being coated on both sides with a permanently adhesive layer. The layers are, for example, those based on a (preferably aqueous) polyacrylate dispersion. According to the present invention, the contact surface of the pressure aid is anti-adhesive with respect to the support side of the adhesive mat. For this purpose, the contact surface comprises or consists of a material that does not adhere or only slightly adheres to the support side of the adhesive mat. As a result, during the application of the adhesive mat, the operator can lift the pressure aid from the adhesive mat by himself without the aid of tools and without having to moisten the adhesive mat and/or the contact surface. “Anti-adhesive” in the context of the present invention means, in particular, that the contact surface is free of any material adhering to the adhesive mat after the pressure aid has been lifted off the adhesive mat. Instead of having to press the adhesive mat onto the shaking platform with bare fingers or fingers wearing a glove and smoothing it out, an operator now contacts the support side of the adhesive mat exclusively via the contact surface of the pressure aid and thus presses it against the shaking platform. Since the contact surface is anti-adhesive with respect to the support side, it is easy to smooth the adhesive mat on the shaking platform without forming wrinkles or leaving air bubbles trapped between the adhesive mat and the shaking platform.

The set according to the present invention has a number of advantages. On the one hand, the adhesive mat, and, in particular, the support side thereof, does not come into contact with materials adhering to fingers or gloves, such as fats, oils, or dust particles; on the other hand, no pressure-sensitive adhesive remains on the operator's fingers. In addition, a more uniform and higher pressing force can be exerted on the adhesive mat via the pressure aid than with bare hands, which, on the one hand, improves the adhesion of the adhesive mat to the shaking platform and, on the other hand, accelerates the attachment. Furthermore, by using the pressure aid, upstream moistening or watering of the adhesive mat can also be dispensed with. The contact surface of the pressure aid practically does not adhere to the non-moistened adhesive mat. This also increases the adhesion of the adhesive mat to the shaking platform. In addition, there is no longer a waiting time after the attachment of the adhesive mat, which in the prior art was necessary until the adhesive mat was dried. After a much easier and faster attachment of the adhesive mat to the shaking platform, a laboratory shaker or shaking incubator can immediately be loaded with containers and put into operation. By putting together an adhesive mat and an associated pressure aid as a set, an operator has all the materials at hand for quick and easy attachment of the adhesive mat on a shaking platform.

As already described, the contact surface of the pressure aid is anti-adhesive with respect to the support side of the adhesive mat, the material of the contact surface itself being anti-adhesive without any further measures. The contact surface is therefore not anti-adhesive because it is moistened with a liquid such as a solvent or water, for example, before use. In particular, the dry, non-moistened contact surface is therefore anti-adhesive. Suitable materials for the contact surface are preferably selected from at least one of polytetrafluoroethylene (PTFE), ethylene-tetrafluoroethylene (ETFE), and silicone, the contact surface comprising at least one of these materials and, in particular, consisting of at least one of these materials. Alternatively, other materials can be used whose adhesive force is in the range of that of the aforementioned materials. It is sufficient if only the contact surface consists of the anti-adhesive material, while the remaining parts of the pressure aid can consist of other materials.

In principle, the pressure aid or the contact surface can have any suitable shape that is suitable for securely attaching the adhesive mat to the shaking platform. For example, the contact surface can be planar and used like a stamp to press on the adhesive mat. A particularly simple pressing on of the adhesive mat is achieved, however, if the contact surface is designed to be at least partially rounded. The contact surface can then be pressed on the support side of the adhesive mat and unrolled. In particular, the rolling movement makes it possible to fix the adhesive mat particularly firmly to the shaking platform and thereby prevent the inclusion of air bubbles between the adhesive mat and the shaking platform and the formation of wrinkles.

The pressure aid can therefore be, for example, a stamp having a planar or preferably rounded, for example semi-cylindrical, contact surface. Pressure aids of this type are sufficient, in particular, in this case of small adhesive mats. Above all when using larger adhesive mats, however, it is preferred that the pressure aid comprises a roll, the outer circumferential surface of which (roll circumference) is the contact surface. In the simplest case, the pressure aid is designed only as a roll body. An operator then presses directly on the roll body with his hand and rolls the roll over the support side of the adhesive mat in order to fix it on the shaking platform. However, handling is even easier if, according to one embodiment of the present invention, the pressure aid is designed as a hand-held roller having a rotatably mounted roll, the roll circumference being the contact surface. A roller is characterized in that the position of the operator's hand relative to the contact surface, apart from the rotational positions of the roll having the contact surface, remains the same during the rolling over the support side of the adhesive mat. This means that even large-area adhesive mats can be quickly and conveniently pressed on a shaking platform and thus fixed.

In order to simplify the handling of the pressure aid, it may have one or two handles. Using the handle or handles, it is particularly easy for an operator to exert a large contact pressure on the adhesive mat and therefore to attach it to the shaking platform in a particularly reliable manner and free of air bubbles and wrinkles. In an embodiment comprising a handle, this can be arranged, for example, transversely to the axis of rotation of the roll of the roller, the handle being connected to the axis of rotation of the roll via a bracket. This embodiment is similar in its structure, for example, to rollers such as those used for painting walls. An alternative embodiment provides, for example, two handles which are arranged as an extension of the axis of rotation of the roll on both sides of the roll on the respective end faces. This embodiment is similar in structure to a rolling pin. For easier handling, the two handles of this embodiment can also be bent, for example in such a way that they both point in the same direction, in particular transversely to the axis of rotation of the roll.

Generic adhesive mats are available in different sizes, for example to cover the different sizes of the shaking platforms of different laboratory shakers and shaking incubators. In order to allow for quick and easy installation of each adhesive mat, it is therefore preferred that the pressure aid, which is provided with each adhesive mat in the set, is adjusted to the size of the adhesive mat. At the same time, it must be taken into account that adhesive mats are also cut to size by the operators. For example, adhesive mats are divided up in order to provide only part of the shaking platform with the adhesive mat, while further regions remain free for receiving other holders. The pressure aid and, in particular, the width of the contact surface should therefore be smaller than the adhesive mat, specifically the width or length thereof, so that parts of the adhesive mat can still be conveniently attached using the pressure aid, and the pressure aid is easy to handle. In another embodiment of the present invention, a width of the contact surface of the pressure aid is substantially half as large as a length or a width of the adhesive mat. The width of the contact surface relates to the extension of the contact surface along the axis of rotation of the roll of the pressure aid. It substantially corresponds to the line with which the outer circumferential surface of the roll rests on the adhesive mat when it is pressed on. The length and the width of the adhesive mat in turn relate to the dimensions of the adhesive mat as provided in the set, i.e., before it is possibly cut to size by an operator. In one embodiment of the present invention the width of the contact surface is between 25% and 75%, preferably between 30% and 70%, particularly preferably between 35% and 65%, very particularly preferably between 40% and 60%, in particular between 45% and 55%, for example 50%, of the length or the width of the adhesive mat. Both the entire adhesive mat and cut-to-size sections thereof can be quickly and easily attached to the shaking platform with a pressure aid of this type.

The above described aspect of the present invention is also achieved by the use of a pressure aid described above for the attachment of an adhesive mat to a shaking platform of a laboratory shaker or shaking incubator. The solution is also achieved using a method described in more detail below. The features, effects, and advantages of the set according to the present invention already described above, and, in particular, of the pressure aid, apply accordingly both to the use and to the method. To avoid repetition, reference is made to the relevant embodiments.

The method according to the present invention for attaching an adhesive mat to a shaking platform of a laboratory shaker or shaking incubator comprises the steps of providing the adhesive mat; placing the adhesive mat on the shaking platform and fixing the adhesive mat by pressing it on using a pressure aid according to the above-described embodiments. Providing the adhesive mat comprises both the procurement of a new adhesive mat, for example as part of a set according to the present invention, and the cleaning of an existing adhesive mat.

Both in the use according to the present invention and in the method according to the present invention, it is preferably provided that the adhesive mat is placed on the shaking platform in a non-moistened or dry state and is also pressed on in a non-moistened or dry state using the pressure aid. Moistening, in particular watering, the adhesive mat is possible, but not necessary, since this can worsen the adhesion to the shaking platform and increase the time required until the adhesive mat can be used as intended. The use and method can in principle be used with all types of laboratory shakers and shaking incubators, for example the devices described in the introduction to the description and the devices described in the patent publications cited.

### BRIEF DESCRIPTION OF THE DRAWINGS

The present invention is described in more detail below with reference to the embodiments shown in the figures. The embodiments serve only to describe preferred embodiments, without the present invention being restricted thereto. Identical or identically acting components are identified in the figures with the same reference signs. Repeated components are not identified separately in each figure. Schematically, in the drawings:

FIG. 1 is a perspective view of a laboratory shaker;

FIG. 2 is a perspective view of an adhesive mat;

FIG. 3 shows an embodiment of a pressure aid as a roll without handles;

FIG. 4 shows an embodiment of a pressure aid as a roller having two handles;

FIG. 5 shows an embodiment of a pressure aid as a roller having a handle;

FIG. 6 shows an embodiment of a pressure aid as a stamp;

FIG. 7 shows the attachment of an adhesive mat to the shaking platform of a laboratory shaker;

FIG. 8 shows the attachment of a cut-to-size adhesive mat to the shaking platform of a laboratory shaker;

FIG. 9 is a perspective view of a shaking incubator; and

FIG. 10 is a flow chart of the method.

### DETAILED DESCRIPTION OF THE INVENTION

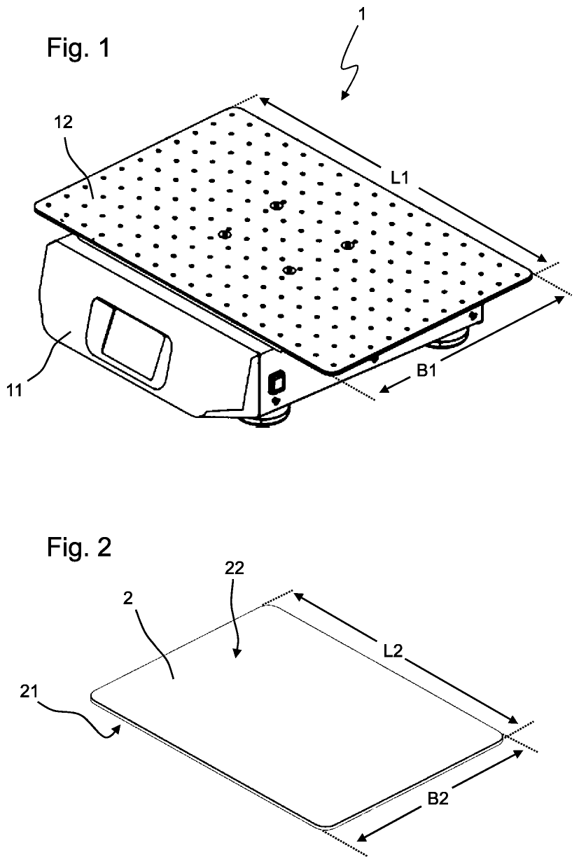

FIG. 1 shows a laboratory shaker 1 having a housing part 11 and a shaking platform 12. In the housing part 11, for example, control electronics and a drive motor are accommodated, which are used to set the shaking platform 12 in shaking movements when the laboratory shaker 1 is in operation. In this way, for example, liquids that are in containers that are placed on the shaking platform 12 are mixed and kept in movement. The shaking platform 12 has a length L1 and a width B1, which are dimensioned differently depending on the size of the device and the application.

FIG. 2 shows an adhesive mat 2. The adhesive mat 2 is designed as a flat, few-millimeters-thin base and may consist of resilient plastics material, the two surfaces 21 and 22 of which are designed to be permanently adhesive. Either the material of the adhesive mat 2 itself is already adhesive or the adhesive mat 2 is coated with an adhesive material, for example a polyacrylate-based pressure-sensitive adhesive. The adhesive mat 2 comprises a platform side 21 or underside which is designed to come into contact with the shaking platform 12 and to adhere thereto. In addition, the adhesive mat 2 has a support side 22 or upper side which is designed to fix containers or the like that are placed thereon. The platform side 21 and the support side 22 are made from the same material and have the same adhesive force. It is, however, also possible for the platform side 21 and the support side 22 to be of different adhesive design. The adhesive mat 2 has a length L2 and a width B2, the dimensions of which are adjusted to the size of the shaking platform 12 of that laboratory shaker 1 for which the adhesive mat 2 is designed. Typically, the length L2 and the width B2 of the adhesive mat 2 substantially correspond to the length L1 and the width B1 of the shaking platform 12 or are slightly smaller than these.

FIGS. 3 to 6 show different embodiments of a pressure aid 3, which together with one or more adhesive mats form a set according to the present invention. In the embodiment according to FIG. 3, the pressure aid 3 is designed as a roll 35 without handles. The outer circumferential surface of the roll 35 forms the contact surface 31, which is designed to contact the support side 22 of the adhesive mat 2 during the attachment of the adhesive mat 2 to the shaking platform 12 and to roll it off on the latter. The contact surface 31 is therefore designed to be anti-adhesive with respect to the adhesive mat 2 and, in particular, the support side 22 thereof. For example, the contact surface 31 consists of PTFE, silicone, or ETFE. The roll 35 can consist entirely of one of the materials described. Alternatively, the core of the roll consists of a different material and is surrounded with a coating or a cover made from PTFE, ETFE, or silicone. For the attachment of the adhesive mat 2 to the shaking platform 12, the adhesive mat is placed with the platform side 21 down on the shaking platform 12, the roll 35 is placed on the support side 22 of the adhesive mat 2 and rolled over the surface thereof, causing the roll 35 to rotate around the axis of rotation R, to press on the adhesive mat against the shaking platform, and to fix it thereon. For better handling, however, it is preferred that the pressure aid 3 has at least one handle.

An embodiment of this type is shown in FIG. 4. In this case, the pressure aid 3 has two handles 32. The handles 32 lie on the ends of a rod which is guided through a longitudinal bore of the roll enclosing the axis of rotation R and on which the roll 35 is rotatably mounted. An operator grabs the pressure aid 3 by the two handles 32 and guides it over the support side 22 of the adhesive mat 2 such that only the contact surface 31 comes into contact therewith. At the same time, with the aid of the handles 32, pressure can be exerted on the adhesive mat 2 in order to fix it securely to the shaking platform 12.

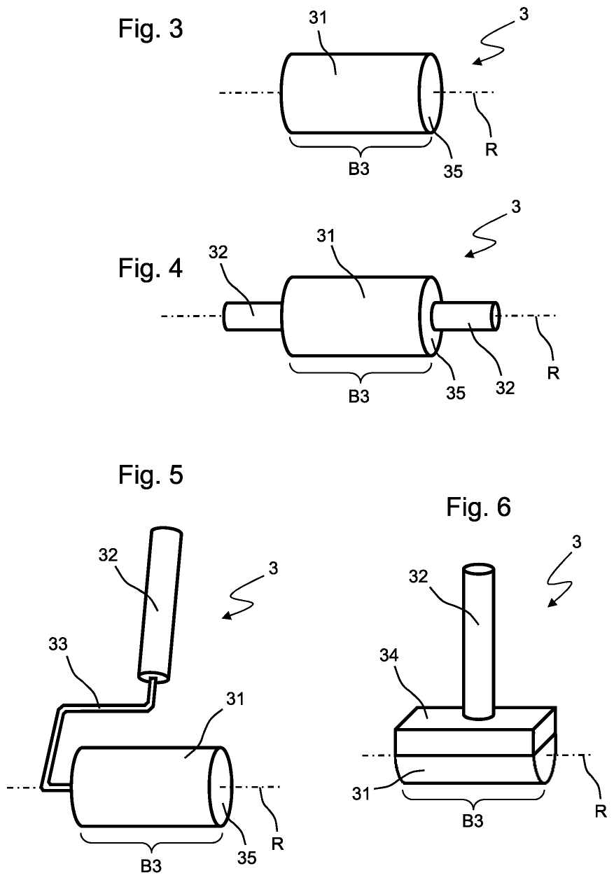

In one embodiment of the present invention the pressure aid 3 is shown in FIG. 5. The corresponding pressure aid 3 is designed as a roller having a handle 32. The handle 32 is arranged substantially perpendicular to the axis of rotation R. The roll 35 is rotatably mounted on one arm of a curved bracket 33, at the other end of which the handle 32 is seated. The roller can thus be guided over the adhesive mat 2 with just one hand.

FIG. 6 shows a further embodiment of the pressure aid 3, which is designed as a stamp in this case. It has a handle 32 which is connected to a stamp base body 34. The stamp base body 34, in turn, has a rounded contact surface 31 which, in the embodiment shown, is designed as a cylinder portion cut parallel to the axis of rotation R. For the attachment of the adhesive mat 2 to the shaking platform 12, the operator grabs the handle 32, presses the contact surface 31 onto the support side 22 of the adhesive mat 2 and performs rocking movements about the axis of rotation R. As a result, the adhesive mat 2 is pressed against the shaking platform 12 and any air bubbles that may be trapped between the two are pressed out.

All embodiments of the pressure aid 3 have in common that the contact surface 31 is anti-adhesive with respect to the support side 22 of the adhesive mat 2. The pressure aid can therefore be lifted off the adhesive mat without difficulty, and no material of the adhesive mat remains adhering to the contact surface 31. The width B3 of the contact surface 31, parallel to the axis of rotation R, is adjusted to the length L2 and/or the width B2 of the adhesive mat 2. For example, the width B3 of the contact surface 31 is substantially half as large as the length L2 or the width B2 of the adhesive mat 2. In this way, both the entire adhesive mat 2 and parts thereof can be quickly and easily attached to the shaking platform 12 using the same pressure aid 3.

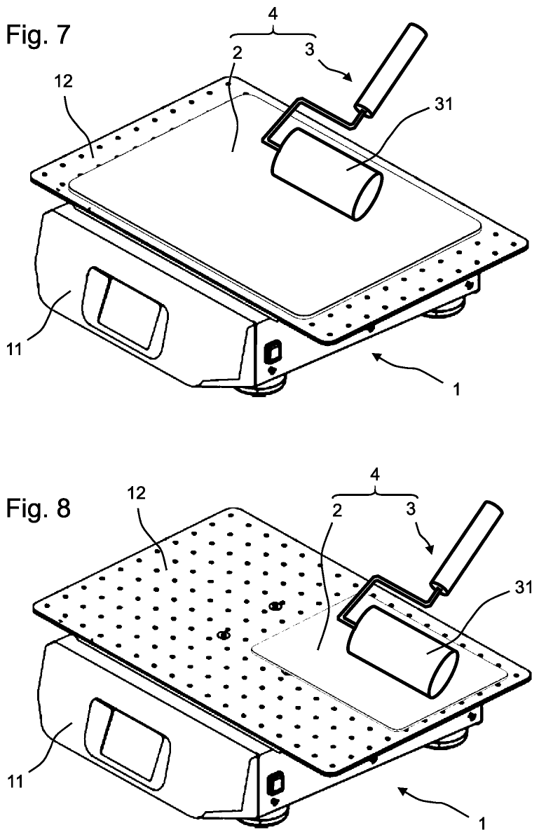

The attachment of an adhesive mat 2 to a shaking platform 12 is illustrated in FIG. 7. Specifically, the adhesive mat 2, which is in a dry, non-moistened state, is placed with the platform side 21 thereof on the shaking platform 12 and is now pressed using the pressure aid 3 by means of rollers, so that the adhesive mat 2 is fixed smoothly and straight on the shaking platform 12. FIG. 8 likewise shows the pressing on of an adhesive mat 2 on a shaking platform 12, with only a partial area of the shaking platform 12 now being covered by the adhesive mat. The rest of the platform can be used, for example, for the attachment of other retaining means (not shown) such as piston clamps. The adhesive mat 2 used in FIG. 8 is either smaller from the start than that of FIG. 7 or is separated from a larger adhesive mat 2 as required. The attachment takes place as described using the pressure aid 3 of the set 4, the width B3 of which is dimensioned such that it can also be used for parts of the adhesive mat 2.

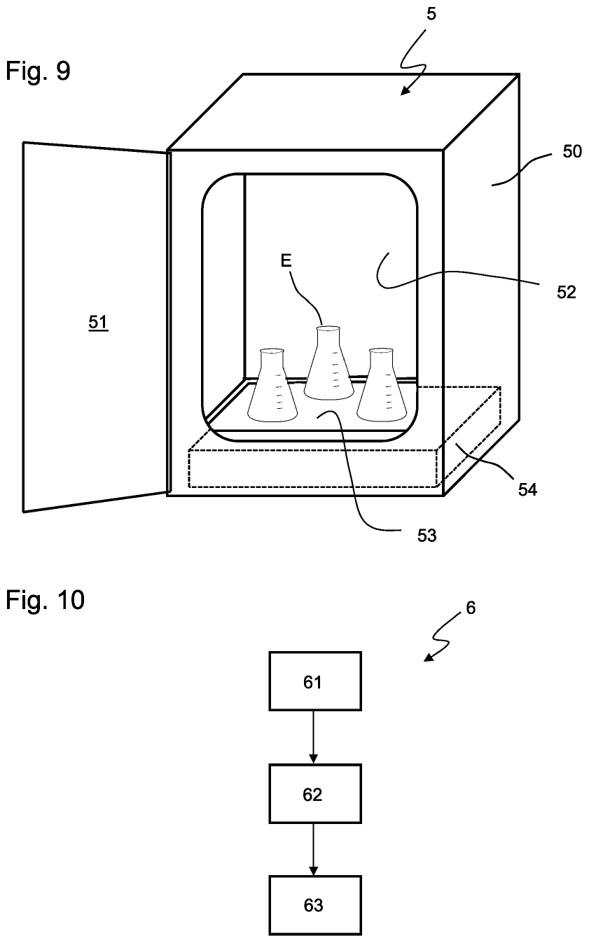

FIG. 9 shows a shaking incubator 5 which differs from the laboratory shaker 1 according to FIG. 1 substantially by a housing 50 which surrounds an interior 52 which can be closed by a door 51, in which a shaking platform 53. On the shaking platform 53, an adhesive mat—not shown separately in this case—is arranged on which some Erlenmeyer flasks E are in turn located. The shaking platform 52 is moved by means of a drive device 54 arranged in this case in the floor region of the incubator. In the interior 52, a desired temperature and/or a desired composition of the interior atmosphere, such as, for example, a predetermined air humidity, can be set in a manner known per se. To set up the sample vessels, instead of one, there can also be a plurality of shaking platforms in the interior, which are also set in motion by the drive device 54.

FIG. 10 shows a flow chart of the method 6 for attaching an adhesive mat 2 to a shaking platform 12. The method 6 begins with the providing 61 of the adhesive mat 2. In this case, for example, an adhesive mat 2 from a newly acquired set 4 can be used. Alternatively, it is also possible to detach an already used adhesive mat 2 from a shaking platform, to clean the adhesive mat 2 and, preferably, to dry it. Next, the placing 62 of the adhesive mat 2 onto the shaking platform 12 takes place. In this case, the platform side 21 of the adhesive mat 2 is brought into contact with the shaking platform 12. Fixing 63 the adhesive mat 2 by pressing it using the pressure aid 3 takes place, for which the pressure aid 3 is guided with its contact surface 31 over the support side 22 of the adhesive mat 2. In the method 6 according to the present invention, the adhesive mat 2 is preferably placed on the shaking platform 12 without having been previously moistened. As a result of the dry pressing and skipping the moistening of the adhesive mat 2, it can be used as a base for setting up vessels immediately after the method 6 has been completed. The waiting times customary in the prior art for the adhesive mat to dry do not apply. In addition, contamination of the support side 22 is avoided since it is only in contact with the contact surface 31 of the pressure aid 3.

What is claimed is:

1. A set comprising an adhesive mat for a shaking platform of a laboratory shaker or shaking incubator, the adhesive mat having a platform side for application to the shaking platform and a support side opposite the platform side, both of which are designed to be adhesive,
wherein the set comprises a pressure aid which has a contact surface which is anti-adhesive with respect to the support side of the adhesive mat.

2. The set according to claim 1,
wherein the contact surface comprises at least one of polytetrafluoroethylene (PTFE), ethylene-tetrafluoroethylene (ETFE), and silicone.

3. The set according to claim 1,
wherein the contact surface is designed to be at least partially rounded.

4. The set according to claim 1,
wherein the pressure aid is designed as a stamp.

5. The set according to claim 1,
wherein the pressure aid is designed as a hand-held roller with a rotatably mounted roll, the roll circumference being the contact surface.

6. The set according to claim 1,
wherein in that the pressure aid has one or two handles.

7. The set according to claim 1,
wherein a width (B3) of the contact surface is between 25% and 75% of a length (L2) or a width (B2) of the adhesive mat.

8. (canceled)

9. A method for attaching an adhesive mat to a shaking platform of a laboratory shaker or shaking incubator, comprising the steps of:
a) providing the adhesive mat;
b) placing the adhesive mat onto the shaking platform, and
c) fixing the adhesive mat by pressing the adhesive mat on the shaking platform via a pressure aid as defined in claim 1.

10. The method according to claim 9,
wherein the adhesive mat is placed on the shaking platform without being moistened and is also pressed on using the pressure aid while the pressure aid is not moistened.

11. The set according to claim 1,
wherein a width (B3) of the contact surface is between 30% and 70% of a length (L2) or a width (B2) of the adhesive mat.

12. The set according to claim 1,
wherein a width (B3) of the contact surface is between 35% and 65% of a length (L2) or a width (B2) of the adhesive mat.

13. The set according to claim 1,
wherein a width (B3) of the contact surface is between 40% and 60% of a length (L2) or a width (B2) of the adhesive mat.

14. The set according to claim 1,
wherein a width (B3) of the contact surface is between 45% and 55% of a length (L2) or a width (B2) of the adhesive mat.

15. The set according to claim 1,
wherein a width (B3) of the contact surface is 50% of a length (L2) or a width (B2) of the adhesive mat.
